(19) United States
(12) Patent Application Publication
(10) Pub. No.: US 2010/0234577 A1
(43) Pub. Date: Sep. 16, 2010

(54) METHODS FOR PURIFYING ANTIBODIES USING CERAMIC HYDROXYAPATITE

(75) Inventors: Gregory J. Mazzola, King of Prussia, PA (US); Thomas M. Smith, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: SMITHKLINE BEECHAM CORPORATION

(21) Appl. No.: 12/304,782

(22) PCT Filed: Jun. 13, 2007
(86) PCT No.: PCT/US07/71055

§ 371 Date: Dec. 15, 2008

Related U.S. Application Data
(60) Provisional application No. 60/804,722, filed on Jun. 14, 2006.
(60) Provisional application No. 60/869,445, filed on Dec. 11, 2006.

(51) Int. Cl. C07K 1/22; C07K 16/00

(52) U.S. Cl. 530/388.1; 530/387.1

(57) ABSTRACT

This invention relates to the purification of monoclonal antibodies from mammalian cell culture fluid utilizing sequential, orthogonal chromatography and filtration techniques resulting in material of high purity and quality that is suitable for human administration. The method involves capturing an IgG product using immobilized protein A affinity chromatography, followed by at least one ion exchange technique prior to adsorbing the IgG to hydroxyapatite and selectively eluting the product in a single isocratic step to achieve purification from impurities and simultaneously reducing multiple types of impurities including but not limited to IgG aggregates, residual protein A, non-IgG proteins, host cell proteins, viral particles, and DNA

### FIELD OF THE INVENTION

This invention relates to the purification of antibodies from cell culture fluids utilizing sequential, orthogonal chromatography and filtration techniques resulting in material of high purity, and quality that is suitable for veterinary and human administration.

### BACKGROUND OF THE INVENTION

When antibodies are produced for therapeutic use, it is necessary to employ proven methods for the reduction of immunogenic, toxic, or otherwise harmful impurities and/or contaminants to levels deemed safe by governing regulatory authorities.

Affinity chromatography using immobilized protein A is a commonly used method for the purification of antibodies, including those intended for clinical manufacture. See, for example, Hahn, R, Schlegel, R, Jungbauer, A. 2003 Comparison of protein A affinity sorbents, Journal of Chromatography B, 790 35-51. The high avidity and specificity for the IgG Fc region makes it one of the most useful tools for the initial isolation of antibodies form cell culture fluid. Although selective elution from the media using low pH buffers often results in 90-95% purity, further purification is necessary in order to remove the final 5-10% of contaminants. In addition, some residual protein A, which is potentially toxic, leaches from the chromatography media. What makes separation of this protein A especially challenging is that it exists as an IgG:Protein A complex which must be separated from the IgG of interest. As useful as protein A chromatography is, it does not provide IgG of high enough quality for administration to humans, being contaminated with the above mentioned impurities.

Aside from the cost of the protein A media, the vast majority of the cost to purify any antibody will be expended toward removing the remaining impurities subsequent to the capture step, which involves multiple additional chromatography steps.

Aside from chromatography steps, there are multiple, orthogonal steps that are necessary for any monoclonal antibody process to give some assurance of viral safety. These are included at strategic places in the purification scheme. These include such things as low pH treatment, viral filtration (sometimes called nanofiltration), heat treatment and chemical inactivation.

Adsorption chromatography on hydroxyapatite is sometimes a very effective process for purification of proteins. See, for example, Tiselius, A., Hjerten, S., Levin O. 1956. Protein chromatography on calcium phosphate columns. Arch. Biochem. Biophys. 65, 132-155. Unlike adsorptive chemistries where a reactive ligand is affixed to a “neutral” matrix, hydroxyapatite is both the ligand and the matrix. Its unit formula is Ca₁₀(PO₄)₆(OH)₂. The introduction of ceramic forms of hydroxyapatite has made it practical as production chromatography media with adequate flow rate and chemical stability characteristics. See, for example, Cummings, L. J., Ogawa, T., Tunon, P. Macro-Prep Ceramic Hydroxyapatite—new life for an old chromatographic technique. Bio-Rad technical bulletin 1927, RevA. Bio-Rad Laboratories. With the exception of chelating agents and pH below about 5-6, hydroxyapatite media are resistant to the harshest cleaning agents, including concentrated sodium hydroxide, urea, guanidine, organic solvents and detergents.

Due to the dual functionality of calcium and phosphate groups comprising the matrix, the specific nature of protein interaction is complex. The amino groups on a protein are attracted to the phosphate sites, but are repelled by calcium sites. The situation is reversed for carboxylic groups, as they are attracted to the calcium sites, but repelled by the phosphate sites. Although amine-binding to phosphate sites and the initial attraction of carboxylic groups to calcium sites are electrostatic, the actual binding of carboxyl groups to calcium sites involves formation of much stronger coordination complex between calcium sites and clusters of protein carboxyls. See, for example, Gorbunoff, M. J. The Interaction of Proteins with Hydroxyapatite: II Role of Acidic and Basic Groups. 1984. Analytical Biochemistry. 136, 433-439.

Once bound, the most common elution mechanism has been a gradient of increasing phosphate concentration. See, for example, Schroder, E., Jonsson, T., Poole, L. 2003. Hydroxyapatite chromatography: altering the phosphate-dependent elution profile of protein as a function of pH. Analytical Biochemistry. 313, 176-178. This would seem to be the most convenient choice of elution buffers since it serves as a displacement agent, disrupting both the COO⁻: Ca⁺ interaction as well as the NH₃⁺: PO₄⁻ interaction. In the increasing phosphate concentration method, all proteins bound to the column can be eluted and resolved based on the strength of the interaction with the phosphate group on the hydroxyapatite matrix. Therefore, by using a gradient of increasing phosphate concentration, the most weakly bound proteins bound by the NH₃⁺: PO₄⁻ interaction (more acidic proteins) will elute earlier than those bound by the COO⁻: Ca⁺ interaction (more basic proteins). In addition, the elution time of various bound proteins, and the resolution between them can be altered significantly through changes in the pH of the elution buffer. See, for example, Schroder, E., Jonsson, T., Poole, L. 2003. Hydroxyapatite chromatography: altering the phosphate-dependent elution profile of protein as a function of pH. Analytical Biochemistry. 313, 176-178. It should be noted that in this elution regimen, impurities which are phosphate bound also elute, and can co-elute with the product of interest.

Elution of proteins by different salts follows different mechanisms. The use of salts such as NaCl (or CaCl or MgCl) act by a charge screening mechanism and by Na (or K, Ca, Mg) displacement by complexing with the resin phosphate groups. Using a gradient of increasing salt concentration, the most basic proteins would elute in lowest concentrations of salt, while acidic proteins are retained even at very high NaCl concentrations. See, for example, Gorbunoff, M. J. Protein Chromatography on Hydroxyapatite Columns. 1985. Methods in Enzymology. 182, 329-339 and Guerrier, L., Flayeux, I., Boschetti, E. 2001. A dual-mode approach to the selective separation of antibodies and their fragments. Journal of Chromatography B. 755. 37-46. Unfortunately, gradient elutions are both costly and difficult to implement reliably in commercial manufacturing environments.

Others have shown some utility in using hydroxyapatite to bind IgG:Protein A complexes and host cell derived proteins while allowing the protein of interest (recombinant fusion proteins, antibodies and TNFR:Fc) to flow through in the non-bound fraction of the purification. See, for example, Vendantham, G., Brooks, Clayton A., Reeder, J. M., Goetze, A. M. 2003. Methods for Purifying Protein. U.S. Patent Application US2003/0166869 A1 and Vendantham, G., Brooks, Clayton A., Reeder, J. M., Goetze, A. M. 2003. Purifying a protein from a sample comprising a protein and at least one protein contaminant comprises subjecting the sample to hydroxyapatite chromatography. International Patent Application WO 2003069935 A2. Additionally, effective separation of IgG from IgG:Protein A complexes has been demonstrated by binding of the sample to ceramic hydroxyapatite (cHA), followed by elution with a phosphate gradient. See, for example, Horenstein, A. L., Crivellin, F., Funaro, A., Said, M., Malavasi, F. 2003. Design and scaleup of downstream processing of monoclonal antibodies for cancer therapy: from research to clinical proof of principal. Journal of Immunological Methods. 275. 99-112.

Various antibodies have also been purified via a secondary or tertiary purification using hydroxyapatite. The primary goal was the separation of IgG monomer from product aggregates. Utilizing a gradient elution of increasing citric acid concentration, separation of both IgG:Protein A complexes and IgG aggregates was achieved. Under other conditions, a step elution was shown to remove aggregates, host cell proteins and DNA, but not IgG:Protein A complexes. See, for example, Ahmad, Z., Scott, R., Diener, A., Smith, T. M., Misczak, J., Wilson, E., Wang, W. K., Nishikawa, A. H., Shadle, P. 1999. SmithKline Beecham Pharmaceuticals. Abstract presented at “Recovery of Biological Products IX” conference.

A limitation of the repeated re-use of hydroxyapatite is the instability of hydroxyapatite in calcium chelating buffers, such as citrate. Therefore, the present invention implements an elution from Protein A using sodium phosphate at pH 2.1-3.5. In this way, the present invention avoids the use of citric acid, allowing direct loading onto hydroxyapatite or alternatively, filtration through an anion-exchange filter, followed by direct loading onto a cHA column. In addition, an embodiment of the present invention uses a purification process in which product comes in direct contact with only sodium phosphate buffers, achieving a high degree of purification by altering only the pH and salt concentrations throughout the entire process.

### SUMMARY OF THE INVENTION

The present invention relates to methods for purifying monoclonal antibodies suitable for human or veterinary administration.

Further, the method of the invention may be applied to traditional purification methodologies to increase the yields of the traditional separations and to render those traditional methods suitably clean to allow for reuse and decontamination of affinity and/or filtration media as well as apparatus or device surfaces used in such purifications. This invention is not to be limited in scope by the specific embodiments described herein. Indeed, various modifications of the invention in addition to those described herein will become apparent to those skilled in the art from the foregoing description. Such modifications are intended to fall within the scope of the appended claims.

Although one embodiment is adapted to the purification of monoclonal antibodies, it may also be used for the purification of other antibodies, for example, polyclonal antibodies, or fragments of monoclonal or polyclonal antibodies.

In one embodiment, the invention relates to a method of purifying antibody in an aqueous solution, such method utilizes an antibody-containing solution, ceramic hydroxyapatite chromatography media, and an isocratic elution. The method of purifying antibody comprises a) applying an antibody-containing solution to ceramic hydroxyapatite chromatography media, wherein the antibody is adsorbed by the ceramic hydroxyapatite chromatography media; and b) selectively elute the antibody by isocratic elution that simultaneously removes at least one impurity.

In other embodiments, the invention provides for a method wherein the impurities removed are selected from the group of: host cell proteins, host cell nucleic acids, retroviral particles, adventitious viruses, impurities introduced during production, impurities introduced during purification, and aggregated forms of the antibody.

In other embodiments, antibodies capable of being purified by the invention are selected from the group of: a monoclonal antibody, a fragment of monoclonal antibody, a polyclonal antibody, and a fragment of polyclonal antibody. In one embodiment, the antibody purified by the method is a monoclonal antibody.

In another embodiment, the invention relates to a method of purifying a monoclonal antibody in an aqueous solution comprising the steps of:
a) applying a monoclonal antibody-containing solution to an affinity chromatography resin, wherein the monoclonal antibody is adsorbed by the affinity chromatography resin;
b) eluting the monoclonal antibody from the affinity chromatography resin;
c) inactivating viral contaminants by adjusting the monoclonal antibody eluate from step b) to pH 2.5 to 4.5 and/or pH 2.5, pH 3.0, pH 3.5, pH 4.0, and pH 4.5;
d) adjusting the monoclonal antibody eluate from step c) to pH 6.0 to 8.5 and/or pH 6.0, pH 6.5, pH 7.0, pH 7.5, pH 8.0, and pH 8.5;
e) filtering the monoclonal antibody eluate from step d) through a 0.2 um filter;
f) filtering the monoclonal antibody filtrate from step e) through an anion exchange filter or anion exchange chromatography medium;
g) applying the monoclonal antibody filtrate from step f) to ceramic hydroxyapatite, wherein the monoclonal antibody is adsorbed by the ceramic hydroxyapatite; and
h) eluting the monoclonal antibody with an isocratic elution buffer.

This embodiment may be sufficient to provide adequate assurance of viral clearance. However, regulatory guidelines may require additional steps to provide adequate assurance of viral clearance.

In another embodiment, the invention relates to a method of purifying a monoclonal antibody in an aqueous solution comprising the steps of:
a) applying a monoclonal antibody-containing solution to an affinity chromatography resin, wherein the monoclonal antibody is adsorbed by the affinity chromatography resin;
b) eluting the monoclonal antibody from the affinity chromatography resin;
c) inactivating viral contaminants by adjusting the monoclonal antibody eluate from step b) to pH 2.5 to 4.5 and/or pH 2.5, pH 3.0, pH 3.5, pH 4.0, and pH 4.5;
d) adjusting the monoclonal antibody eluate from step c) to pH 6.0 to 8.5 and/or pH 6.0, pH 6.5, pH 7.0, pH 7.5, pH 8.0, and pH 8.5;
e) filtering the monoclonal antibody eluate from step d) through a 0.2 um filter;
f) filtering the monoclonal antibody filtrate from step e) through an anion exchange filter or anion exchange chromatography medium;
g) applying the monoclonal antibody filtrate from step f) to ceramic hydroxyapatite, wherein the monoclonal antibody is adsorbed by the ceramic hydroxyapatite;
h) eluting the monoclonal antibody with an isocratic elution buffer;
i) filtering the monoclonal antibody eluate from step h) through a virus filter; and
j) formulating the monoclonal antibody from step i) by ultrafiltration and continuous diafiltration.

In another embodiment, the invention relates to a method of purifying a monoclonal antibody or fragment thereof in an aqueous solution comprising the steps of:

a) contacting the monoclonal antibody or fragment thereof in an aqueous solution to an affinity chromatography resin containing an immobilized recombinant Protein A ligand;
b) washing the bound monoclonal antibody or fragment thereof from step a) with a wash solution at about pH 7, which does not elute the monoclonal antibody or fragment thereof;
c) eluting the monoclonal antibody or fragment thereof from step b) with an elution buffer at pH 2.1 to 4.0 and/or pH 2.1, pH 2.5, pH 3.0, pH 3.5, and pH 4.0;
d) inactivating viral contaminants by adjusting the monoclonal antibody or fragment thereof eluate from step c) to pH 2.5 to 4.5 and/or pH 2.5, pH 3.0, pH 3.5, pH 4.0, and pH 4.5, with acid for 15 to 60 minutes;
e) adjusting the monoclonal antibody or fragment thereof eluate from step d) to pH 6.0 to 8.5 and/or pH 6.0, pH 6.5, pH 7.0, pH 7.5, pH 8.0, and pH 8.5, with base;
f) filtering the monoclonal antibody or fragment thereof eluate from step e) through a 0.2 um filter;
g) binding the monoclonal antibody or fragment thereof from step f) to ceramic hydroxyapatite;
h) washing the bound monoclonal antibody or fragment thereof from step g) with a wash solution at pH 6.5 to 8.0 and/or pH 6.0, pH 6.5, pH 7.0, pH 7.5, pH 8.0, and pH 8.5, which does not elute the monoclonal antibody or fragment thereof;
i) eluting the monoclonal antibody or fragment thereof from step h) with an isocratic elution buffer at pH 6.5 to 8.0 and/or pH 6.0, pH 6.5, pH 7.0, pH 7.5, and pH 8.0, 10 to 50 mM sodium phosphate and 50 mM to 1.0 M sodium chloride;
j) filtering the monoclonal antibody or fragment thereof eluate from step i) through a virus filter; and
k) formulating the monoclonal antibody or fragment thereof from step j) by ultrafiltration and continuous diafiltration.

In another embodiment, the invention relates to a method of purifying a monoclonal antibody or fragment thereof in an aqueous solution comprising the steps of:

a) contacting the monoclonal antibody or fragment thereof in an aqueous solution to an affinity chromatography resin containing an immobilized recombinant Protein A ligand;
b) washing the bound monoclonal antibody or fragment thereof from step a) with a wash solution at about pH 7, which does not elute the monoclonal antibody or fragment thereof;
c) eluting the monoclonal antibody or fragment thereof from step b) with an elution buffer at pH 2.1 to 4.0 and/or pH 2.1, pH 2.5, pH 3.0, pH 3.5, and pH 4.0;
d) inactivating viral contaminants by adjusting the monoclonal antibody or fragment thereof eluate from step c) to pH 2.5 to 4.5 and/or pH 2.5, pH 3.0, pH 3.5, pH 4.0, and pH 4.5, with acid for 15 to 60 minutes;
e) adjusting the monoclonal antibody or fragment thereof eluate from step d) to pH 6.0 to 8.5 and/or pH 6.0, pH 6.5, pH 7.0, pH 7.5, pH 8.0, and pH 8.5, with base;
f) filtering the monoclonal antibody or fragment thereof eluate from step e) through a 0.2 um filter;
g) filtering the monoclonal antibody or fragment thereof eluate from step f) through an anion exchange filter or anion exchange chromatography medium;
h) binding the monoclonal antibody or fragment thereof from step g) to ceramic hydroxyapatite;
i) washing the bound monoclonal antibody or fragment thereof from step h) with a wash solution at pH 6.5 to 8.0 and/or pH 6.5, pH 7.0, pH 7.5, and pH 8.0, which does not elute the monoclonal antibody or fragment thereof;
j) eluting the monoclonal antibody or fragment thereof from step i) with an isocratic elution buffer at pH 6.5 to 8.0 and/or pH 6.5, pH 7.0, pH 7.5, and pH 8.0, 10 to 50 mM sodium phosphate and 50 mM to 1.0 M sodium chloride;
k) filtering the monoclonal antibody or fragment thereof eluate from step j) through a virus filter; and
l) formulating the monoclonal antibody or fragment thereof from step k) by ultrafiltration and continuous diafiltration.

In yet another embodiment, the invention relates to a method of purifying a monoclonal antibody or fragment thereof in an aqueous solution comprising the steps of:

a) contacting the monoclonal antibody or fragment thereof in an aqueous solution to an affinity chromatography resin containing an immobilized recombinant Protein A ligand;
b) washing the bound monoclonal antibody or fragment thereof from step a) with a wash solution at about pH 7, which does not elute the monoclonal antibody or fragment thereof;
c) eluting the monoclonal antibody or fragment thereof from step b) with about 25 mM or 25 mM citric acid elution buffer at about pH 3.5 or pH 3.5;
d) inactivating viral contaminants by adjusting the monoclonal antibody or fragment thereof eluate from step c) to pH 2.5 to 4.5 and/or pH 2.5, pH 3.0, pH 3.5, pH 4.0, and pH 4.5, with acid for 15 to 60 minutes;
e) adjusting the monoclonal antibody or fragment thereof eluate from step d) to pH 3.5 to 7.5 and/or pH 3.5, pH 4.0, pH 4.5, pH 5.0, pH 5.5, pH 6.0, pH 6.5, pH 7.0, and pH 7.5, with base;
f) filtering the monoclonal antibody or fragment thereof eluate from step e) through a 0.2 um filter;
g) binding the monoclonal antibody or fragment thereof eluate from step f) to a cation exchange chromatography medium;
h) washing the bound monoclonal antibody or fragment thereof from step g) with a wash solution at pH 5.5 to 8.0 and/or pH 5.5, pH 6.0, pH 6.5, pH 7.0, pH 7.5, pH 8.0, and pH 8.5, which does not elute the monoclonal antibody or fragment thereof;
i) eluting the monoclonal antibody or fragment thereof from step h) with an elution buffer at pH 5.5 to 8.0 and/or pH 5.5, pH 6.0, pH 6.5, pH 7.0, pH 7.5, pH 8.0, and pH 8.5, consisting of 10 to 100 mM sodium phosphate and 10 mM to 200 mM sodium chloride;
j) filtering the monoclonal antibody or fragment thereof eluate from step i) through an anion exchange filter or anion exchange chromatography medium;
k) binding the monoclonal antibody or fragment thereof from step j) to ceramic hydroxyapatite;
l) washing the bound monoclonal antibody or fragment thereof from step k) with a wash solution at pH 6.5 to 8.0 and/or pH 6.5, pH 7.0, pH 7.5, and pH 8.0, which does not elute the monoclonal antibody or fragment thereof;
m) eluting the monoclonal antibody or fragment thereof from step 1) with an isocratic elution buffer at pH 6.5 to 8.0 and/or pH 6.5, pH 7.0, pH 7.5, and pH 8.0, 10 to 50 mM sodium phosphate and 50 mM to 1.0 M sodium chloride;
n) filtering the monoclonal antibody or fragment thereof eluate from step m) through a virus filter; and
o) formulating the monoclonal antibody or fragment thereof from step n) by ultrafiltration and continuous diafiltration.

It is to be understood that both the foregoing summary description and the following detailed description are exemplary and explanatory, and are intended to provide further explanation of the invention as claimed.

The accompanying drawings are included to provide a further understanding of the invention, and are incorporated in, and constitute a part of this specification, illustrate several embodiments of the invention, and together with the description serve to explain the principles of the invention.

### BRIEF DESCRIPTION OF THE FIGURES

FIG. 1 graphically illustrates a process that involves monoclonal antibody purification by use of Protein A affinity, a preparative pH adjustment, and cHA chromatography.

FIG. 2 graphically illustrates a process that involves monoclonal antibody purification by use of Protein A affinity, a low pH adjustment for viral inactivation, an additional preparative pH adjustment, cHA chromatography, and final formulation by ultrafiltration/diafiltration.

FIG. 3 graphically illustrates a process that involves monoclonal antibody purification by use of Protein A affinity, a low pH adjustment for viral inactivation, an additional preparative pH adjustment, anion exchange filtration (or chromatography), cHA chromatography, and final formulation by ultrafiltration/diafiltration.

FIG. 4 graphically illustrates a process that involves monoclonal antibody purification by use of Protein A affinity, a low pH adjustment for viral inactivation, an additional preparative pH adjustment, cation exchange chromatography, anion exchange filtration (or chromatography), cHA chromatography, and final formulation by ultrafiltration/diafiltration.

### DETAILED DESCRIPTION OF THE INVENTION

In the description of the present invention, certain terms are used as defined below.

Recombinant proteins such as monoclonal antibodies produced in mammalian host cells are secreted extracellularly into the cell culture media. Harvest of the protein of interest involves separating the intact cells and cellular debris from the cell culture fluid by ultrafiltration or centrifugation. The product obtained from this process is further clarified by filtration and is referred to herein as clarified unconditioned bulk or “CUB”.

“Affinity chromatography” refers to chromatography that utilizes the specific, reversible interactions between biomolecules, for example, the ability of Protein A to bind to an Fc portion of an IgG antibody, rather than the general properties of a molecule, such as isoelectric point, hydrophobicity, or size, to effect chromatographic separation. In practice, affinity chromatography involves using an absorbent, such as Protein A affixed to a solid support, to chromatographically separate molecules that bind more or less tightly to the absorbent.

“Protein A” is a protein originally discovered in the cell wall of Stapphylococcus that binds specifically to an Fc portion of IgG antibody. For purposes of the invention, Protein A is any protein identical or substantially similar to Stapphylococcal Protein A, including commercially available and/or recombinant forms of Protein A. For purposes of the invention, the biological activity of Protein A for the purpose of determining substantial similarity includes the capacity to bind to an Fc portion of IgG antibody.

“Protein G” is a protein originally discovered in the cell wall of Streptococcus that binds specifically to an F C portion of an IgG antibody. For purposes of the invention, Protein G is any protein identical or substantially similar to Streptococcal Protein G, including commercially available and/or recombinant forms of Protein G. For purposes of the invention, the biological activity of Protein G for the purpose of determining substantial similarity includes the capacity to bind to an Fc portion of IgG antibody.

“Protein LG” is a recombinant fusion protein that binds to IgG antibodies comprising portions of both Protein G (see definition above) and Protein L. Protein L was originally isolated from the cell wall of Peptostreptococcus. Protein LG comprises IgG binding domains from both Protein L and G. Vola et al. (1994) Cell. Biophys. 24-25: 27-36, which is incorporated herein in its entirety. For purposes of the invention, Protein LG is any protein identical or substantially similar to Protein LG, including commercially available and/or recombinant forms of Protein LG. For purposes of the invention, the biological activity of Protein LG for the purpose of determining substantial similarity includes the capacity to bind to an IgG antibody.

“Cation exchange resins” refers to an ion exchange resin with covalently bound negatively charged ligands, and which thus has free cations for exchange with cations in a solution with which the resin is contacted. A wide variety of cation exchange resins, for example, those wherein the covalently bound groups are carboxylate or sulfonate, are known in the art. Chromatography and loading of the protein to be purified can occur in a variety of buffers or salts including, but not limited to sodium, potassium, ammonium, magnesium, calcium, chloride, fluoride, acetate, phosphate, and/or citrate salts and/or Tris buffer. One skilled in the art will recognize that the exact composition and pH of the buffers used should be tailored to result in the desired interaction with the target substance.

“Anion exchange resins” refers to an ion exchange resin or filter membrane (such as Mustang Q™ or Intercept Q™) with covalently bound positively charged groups, such as tertiary or quaternary amino groups. Chromatography and loading of the protein to be purified through the column can occur in a variety of buffers or salts including, but not limited to sodium, potassium, ammonium, magnesium, calcium, chloride, fluoride, acetate, phosphate, and/or citrate salts and/or Tris buffer. One skilled in the art will recognize that the exact composition and pH of the buffers used should be tailored to result in the desired interaction with the target substance.

“Hydroxyapatite chromatography” is chromatography using ceramic hydroxyapatite as an absorbent. Chromatography and loading of the protein to be purified can occur in a variety of buffers or salts including, but not limited to sodium, potassium, ammonium, magnesium, calcium, chloride, fluoride, acetate, phosphate, and/or citrate salts and/or Tris buffer. Such buffers or salts can have a pH of at least about 5.5. In some embodiments, equilibration may take place in a solution comprising a Tris or a sodium phosphate buffer. Optionally, the sodium phosphate buffer is at a concentration between about 1 mM and about 50 mM, in another embodiment at a concentration between about 10 mM and 30 mM. In one embodiment, equilibration takes place at a pH of at least about 5.5. In one embodiment, the solution comprises a sodium phosphate buffer at a concentration of about 30 mM and at a pH of about 7.0. Equilibration may take place at pHs between about 6.0 and about 8.6, in another embodiment at pHs between about 6.5 and 7.5. Equilibration buffers may also contain additional salts including sodium, potassium, ammonium, magnesium, calcium, chloride, fluoride at a concentration between about 1 mM and about 50 mM, in another embodiment at a concentration between about 25 mM and 50 mM. The elution buffer, in one embodiment contains the same buffer composition with additional salts (including the types listed above) added to concentrations between 100 mM and 2 M, in another embodiment at a concentration between about 250 millimolar and 1 molar.

“Equilibration liquid” or “equilibration buffer” refers to liquids of appropriate buffering capacity and pH to prepare the chromatography column or charged membrane to effect the desired chemistry of interaction with target substance. Typical equilibration liquids are well known in the chromatography art and can include a variety of buffers or salts including sodium, potassium, ammonium, magnesium, calcium, chloride, fluoride, acetate, phosphate, and/or citrate salts and/or Tris buffer. One skilled in the art will recognize that the exact composition and pH of the buffer used should be tailored to result in the desired interaction with the target substance.

“Wash liquid” or “wash buffer” refers to the liquids used to wash unbound or loosely bound contaminants away from the chromatography resin to which is bound the target substance. Typical equilibration liquids are well known in the chromatography art and can include a variety of buffers or salts including sodium, potassium, ammonium, magnesium, calcium, chloride, fluoride, acetate, phosphate, and/or citrate salts and/or Tris buffer. One skilled in the art will recognize that the exact composition and pH of the buffer used should be tailored to result in the desired interaction with the target substance.

“Elution liquid” or “elution buffer” refers herein to the liquid that is used to dissociate the target substance away from the chromatography resin after it has been cleansed of contaminants. The elution liquid acts to dissociate the target substance without denaturing it irreversibly. Typical elution liquids are well known in the chromatography art and may have higher concentrations of salts, free affinity ligands or analogs, or other substances that promote dissociation of the target substance from the chromatography resin. Elution conditions refers to process conditions imposed on the target substance-bound chromatography resin that dissociate the (undenatured) target substance from the chromatography resin, such as the contacting of the target substance-bound chromatography resin with an elution liquid or elution buffer to produce such dissociation.

“Drug Product” or “Product” that is suitable for human administration refers to a product meeting the quality, purity, and safety standards expected from a process following the current good manufacturing practices (cGMP) outlined by the FDA, ICH, and other governing regulatory authorities. Proteins purified by the methods described here and meeting these stringent requirements should not be considered suitable only for human administration and should therefore not be limited in scope as to the intended final application. Additionally, the embodiments described here illustrate examples in which the intended use is for human administration. However, these embodiments should not be considered limiting, as certain steps outlined throughout these illustrations can be considered optional if the intended use of the protein is not for human administration. For example, an IgG purified by the methods described here and intended to be used as a reagent for biological assay would not necessarily be filtered through an anion exchange filter or a viral filter for viral removal.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Native antibodies” and “immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (k) and lambda (l), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, and IgA₂. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antibody” is used in the broadest sense and specifically covers single monoclonal antibodies (including agonist and antagonist antibodies) and antibody compositions with polyepitopic specificity. In addition, the term “antibody” will be used herein to describe all suitable immunoglobulins and fragments thereof which have suitable specific binding for an antigen to allow their use in an ELISA or ELISA type detection system.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a single-chain antibody fragment or single chain polypeptide), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers taught by Kostelney et al., J. Immunol., 148: 1547-1553 (1992) and the GCN4 leucine zipper described in the Examples below.

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567 to Cabilly et al.). The monoclonal antibodies also include clones of antigen-recognition and binding-site containing antibody fragments (Fv clones) isolated from phage antibody libraries using the techniques described in Clackson et al., Nature, 352:624-628 (1991) and Marks et al., J. Mol. Biol., 222:581-597 (1991), for example.

The monoclonal antibodies herein include “hybrid” and “recombinant antibodies” produced by splicing a variable (including hypervariable) domain of an anti-IL-8 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity. (See, e.g., U.S. Pat. No. 4,816,567 to Cabilly et al.; Mage and Lamoyi, in Monoclonal Antibody Production Techniques and Applications, pp. 79-97 (Marcel Dekker, Inc., New York, 1987).)

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al., Proc. Natl. Acad. Sci. U.S.A. 81:6851 (1984)).

“Humanized” forms of non-human (e.g., murine) antibodies are specific chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Furthermore, humanized antibodies can comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature 321:522 (1986); Reichmann et al., Nature 332:323 (1988); and Presta, Curr. Op. Struct. Biol. 2:593 (1992).

The present invention provides, among other things, methods for purifying monoclonal antibodies suitable for human administration. An embodiment of the invention is the use of ceramic hydroxyapatite (cHA) in the purification of monoclonal antibodies (mAb) to remove multiple classes of impurities simultaneously. These impurities and/or contaminants include but are not limited to: 1) proteins from the recombinant host cells, 2) nucleic acids from the host cells, 3) retroviral particles and adventitious viruses, 4) impurities introduced during production and purification such as medium components, protein A leached during affinity purification, and 5) aggregated forms of the antibody itself. Henceforth, these shall be collectively referred to as impurities or contaminants when used in a general sense, and when not specifying a contaminant or impurity of the type mentioned above.

In a further aspect, the present invention relates to methods of purification of monoclonal antibodies using sequential chromatography and filtration steps. Such steps include ceramic hydroxyapatite to complete the purification process and provide high quality, high purity monoclonal antibody suitable for human administration. There are at least three embodiments of the invention which allow the user to tailor the purification to the intrinsic properties of monoclonal antibodies and still provide material suitable for administration to humans.

For a particular monoclonal antibody, the conditions used for purification over ceramic hydroxyapatite (buffers used and amounts of protein loaded onto the column) can be tailored to exploit the intrinsic properties of monoclonal antibodies while simultaneously maintaining the ability to remove multiple impurities and contaminants in a single processing step.

The instant invention teaches the use of ceramic hydroxyapatite for the removal of impurities or contaminants that include but are not limited to: 1) proteins from the recombinant host cells, 2) nucleic acids from the host cells, 3) retroviral particles and adventitious viruses, 4) impurities introduced during production and purification such as medium components, protein A leached during affinity purification, and 5) aggregated forms of the antibody itself. Furthermore, the instant invention teaches the use of ceramic hydroxyapatite for the removal of the five classes of the residual contaminants listed above by adsorbing IgG (of subclass IgG₁ and IgG₄) and selectively eluting the IgG in a single desorption event (isocratic, without a gradient) in order to achieve purified antibody having a high level of purity and appropriate for human administration. In this way, it is possible to achieve a cost effective, two step column chromatography process to purify monoclonal antibodies from cell culture fluid. Additional chromatography and/or filtration steps can be included at various stages throughout the process for assurance of viral safety and reduction of DNA if necessary, and a final ultrafiltration/diafiltration step is included in order to prepare the protein for administration or lyophilization.

Gradient elutions are both costly and difficult to implement reliably in commercial manufacturing environments. Therefore, as an alternative, the present invention discloses a step elution designed to exploit the dual functionality of this media and make best use of the intrinsic properties of the IgG to be purified, as well as the properties of the impurities to be partitioned from the IgG. For example, knowing that basic and acidic proteins can by eluted by different mechanisms once bound to cHA, one could elute IgG's preferentially by a sodium chloride displacement of the amine-phosphate interaction, leaving more acidic proteins bound via the carboxyl-calcium interaction. The more acidic proteins can be eluted using ions which form stronger complexes with calcium, i.e. phosphate.

By tailoring the pH and ionic strength of the buffers used throughout the chromatography step, the present invention discloses a method to selectively adsorb an IgG of interest, and subsequently elute an IgG, while resolving the IgG away from the contaminants.

The following examples are further illustrative of the present invention. This example is not intended to limit the scope of the present invention, and provides further understanding of the invention.

### EXAMPLES

The invention is further illustrated by way of the following examples which are intended to elucidate the invention. These examples are not intended, nor are they to be construed, as limiting the scope of the invention. Numerous modifications and variations of the present invention are possible in view of the teachings herein and, therefore, are within the scope of the invention. The examples below are carried out using standard techniques, and such standard techniques are well known and routine to those of skill in the art, except where otherwise described in detail.

### Example 1

### Purification of Monoclonal Antibody (an IgG₁, anti-IL-5 Antibody) which Includes Sequential Protein a to Ceramic Hydroxyapatite Chromatography

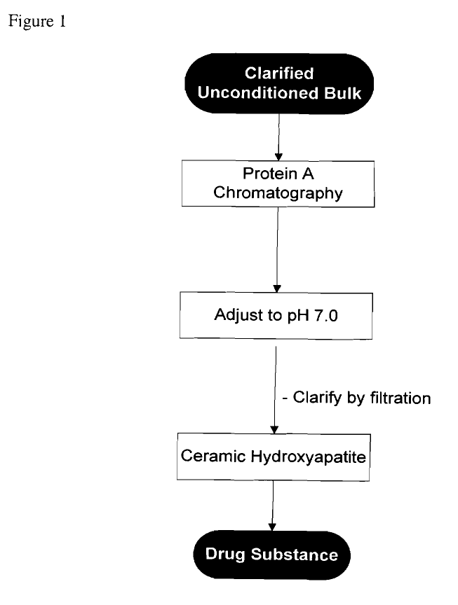

In one embodiment, the process involves antibody purification by use of Protein A affinity, a preparative pH adjustment, and cHA chromatography. This process is depicted in FIG. 1.

By using cHA as described herein, only two chromatography steps are required, reducing the cost of manufacturing without compromising or diminishing the quality, purity or suitability of the monoclonal antibodies used for human administration.

Details of the individual process steps are described below. For each step, a brief description is given including the expected outcome. Important process parameters for each step are listed. All procedural details, buffer compositions, process set-points, column dimensions, etc. are included for illustrative purposes and should not be considered inclusive or restrictive in the operation of this art.

### 1.1 Affinity Chromatography

Monoclonal antibody (for example, an anti-IL-5 antibody described in U.S. Pat. Nos. 5,693,323, 5,683,892, 6,129,913, 5,783,184, and 6,946,130, herein incorporated by reference) captured from filtered cell culture media onto an affinity chromatography resin containing an immobilized recombinant Protein A ligand, MABSELECT™ (protein A media used could also include, but not limited to: Protein A SEPHAROSE™, recombinant Protein A SEPHAROSE™, MABSELECT™, MABSELECT SURE™, MABSELECT XTRA™, PROSEP® A, PROSEP® vA, PROSEP® rA, POROS® 50 A, AF-PROTEIN A TOYOPEARL® 650 M) which had previously been equilibrated with 30 mM phosphate, pH 7.0. Following a salt wash with 3 bed volumes (BV) of a 30 mM phosphate buffer containing 2M sodium chloride, pH 7, and 3 BV of 20 mM Phosphate, pH 5.5, product was eluted with a pH shift using 30 mM sodium phosphate buffer, pH 2.7. One skilled in the art will recognize that buffer composition can include, but is not limited to phosphate, citrate, acetate, etc. in pH ranges of 2.5-3.5. However, the use of phosphate or other non-calcium chelating buffers allows the subsequent cHA column to be used repeatedly without exposure to chelating buffer. Product peak collection started with the rise in UV absorbance and continued until the absorbance peak returned near to baseline. There are no critical peak cutting criteria. The intent is to collect the entire peak, while preventing unnecessary dilution of the product by collecting more eluate after UV absorbance returns close to baseline. For stability purposes, the recommended storage time for unadjusted MABSELECT™ eluate is up to 14 days at 2-10° C. Following elution, the eluate was ≦3.5 and therefore no pH adjustment was necessary. The MABSELECT™ column was stripped with a pH 1.5 hydrochloric and was cleaned with 6 M guanidine in 50 mM sodium phosphate pH 7.0 at the end of the batch. The MABSELECT™ column was stored in 0.1M sodium acetate, 0.5 M sodium chloride containing 1% benzyl alcohol pH 5.2.

MABSELECT™ chromatography removes a large portion of cell and media derived impurities and viral particles. In a novel approach, the salt wash is included to achieve additional DNA clearance. This affinity purification technique results in ≧90% purity of the IgG with yields in excess of 90%.

### 1.2 pH Adjustment and Filtration

As in this example, using elution buffers described above in the 2.5-3.5 pH range, may result in eluate at or below pH 3.5, which, therefore, would not require pH adjustment for the purposes of viral inactiviation. If pH adjustment is necessary, the protein A eluate is adjusted to pH 2.5-4.5 with acid.

Following a 30 minute hold for viral inactivation, eluate was adjusted to pH 7.0 with base. The pH adjusted eluate was then 0.2 um filtered.

The low pH treatment is intended to inactivate potential viral contaminants, especially retroviruses and other enveloped viruses. Filtration at higher pH prepares the solution for the next chromatography step and reduces DNA.

### 1.3 cHA Chromatography

Filtered eluate (from Step 1.2) was further purified by hydroxyapatite chromatography (using any hydroxyapatite resin such as BioRad CHT™ Type I resin or CHT™ Type II resin).

The column was prepared for chromatography by rinsing with 5 BV of pre-equilibration buffer consisting of 400 mM phosphate, pH 7.0. (Alternative buffers to be considered for appropriate use throughout the process include but are not limited to Tris, acetate, MES, etc.) The column was then equilibrated with 5 BV of equilibration buffer consisting of 30 mM phosphate, 50 mM sodium chloride, pH 7.0. The cHA load sample was loaded onto the column. Upon completion of loading, the column was washed with 5 BV of Wash buffer consisting of 30 mM sodium phosphate, 50 mM sodium chloride, pH 7.0. Bound product was eluted with Elution Buffer consisting of 30 mM sodium phosphate and 300, 400, 500 mM sodium chloride, pH 7.0 in 3 separate experiments respectively. Using an on-line UV monitor with 2 mm path length, the peak was collected when absorbance rose to 880 mAU and continued until the peak returns to an absorbance corresponding to 2.0 AU. Peak cutting criteria may be critical for impurity removal and the criteria are expected to be specific for a given monoclonal antibody. Following eluate collection, the column was cleaned with 5 BV of 0.5 N NaOH. Following cleaning the column is washed with 3 BV of 0.01 N NaOH storage solution.

The cHA step was demonstrated to simultaneously removed several impurities such as (but not limited to), host cell proteins, DNA, protein A (protein A:IgG complexes), and IgG aggregates. Results are shown in Table 1 below.

TABLE 1
Example 1 Process Performance

| | | | | Host Cell | |
|---|---|---|---|---|---|
| | | % IgG | ProteinA | Protein | DNA |
| Eluate Sample | Elution Buffer | Aggregate | (ng/mg) | (ppm) | (pg/mg) |
| pH Adjusted | 30 mM Na. Phosphate, | 3.60 | 1.16 | 2.70 | 226.00 |
| Mabselect | pH 2.7 | | | | |
| Eluate | | | | | |
| cHA eluate 1 | 300 mM NaCl, 30 mM | 0.4 | 0.06 | <2.0 | 3.65 |
| | Phosphate, pH 7.0 | | | | |
| cHA eluate 2 | 400 mM NaCl, 30 mM | 0.4 | 0.07 | <2.0 | 12.3 |
| | Phosphate, pH 7.0 | | | | |
| cHA eluate 3 | 500 mM NaCl, 30 mM | 0.30 | 0.05 | <2.0 | 6.80 |
| | Phosphate, pH 7.0 | | | | |

It is important to note that the specific monoclonal antibody used for Example 1, undergoes light-induced aggregation. As a result, all process intermediates were shielded from light with protective shrouding. In experiments where the product was not protected from light, cHA eluate IgG levels ranged from 1.0-3.9%. It is not expected that this is necessary for all antibodies.

### Example 2

### Includes sequential Protein A to Ceramic Hydroxyapatite to Viral Filter and Final Formulation by Ultrafiltration/Diafiltration

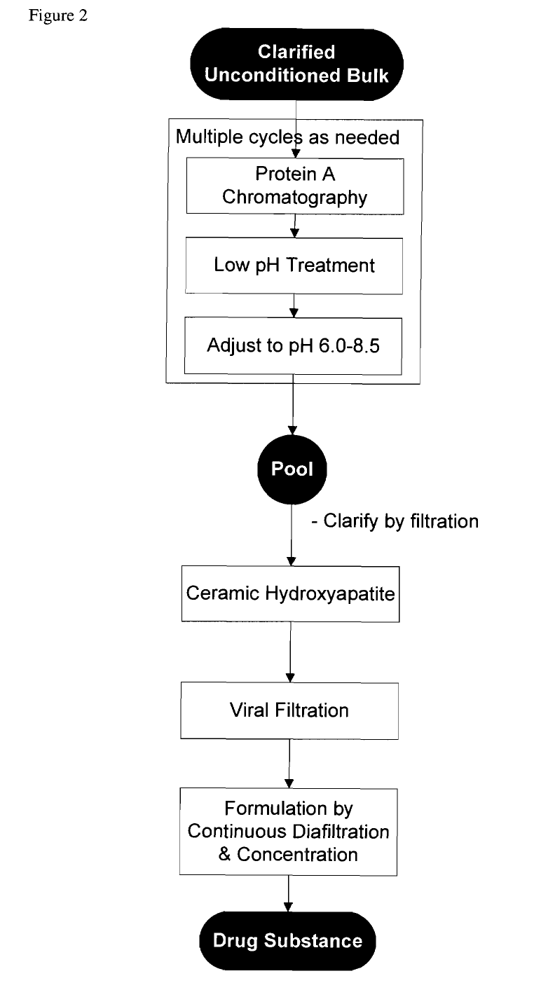

In one embodiment, the process involves antibody purification by use of Protein A affinity, a low pH adjustment for viral inactivation, an additional preparative pH adjustment, cHA chromatography, and final formulation by ultrafiltration/diafiltration. This process is depicted in FIG. 2.

By using cHA as described herein, only two chromatography steps are required, reducing the cost of manufacturing without compromising or diminishing the quality, purity or suitability of the monoclonal antibodies used for human administration.

Details of the individual process steps are described below. For each step, a brief description is given including the expected outcome. Important process parameters for each step are listed. All procedural details, buffer compositions, process set-points, column dimensions, etc. are included for illustrative purposes and should not be considered inclusive or restrictive in the operation of this art.

### 2.2 Affinity Chromatography

Monoclonal antibody (mAb, IgG) is captured from filtered cell culture media onto an affinity chromatography resin containing an immobilized recombinant Protein A ligand (including, but not limited to: Protein A SEPHAROSE™, recombinant Protein A SEPHAROSE™, MABSELECT™, MABSELECT SURE™, MABSELECT XTRA™, PROSEP® A, PROSEP® vA, PROSEP® rA, POROS® 50 A, AF-PROTEIN A TOYOPEARL® 650 M) which had previously been equilibrated with 10-50 mM phosphate, pH 6.0-8.5. Following a salt wash with 2-8 bed volumes (BV) of a 10-50 mM phosphate buffer containing 1-2M sodium chloride, in one embodiment 2M sodium chloride, pH 7.0 and a 3 BV wash with 20 mM sodium phosphate buffer pH 7.0, product is eluted with a pH shift using 10-50 mM sodium phosphate buffer, pH 2.1-3.5. Higher pH (e.g., up to pH 4.0) may be used if the yield is acceptable. One skilled in the art will recognize that buffer composition can include, but is not limited to phosphate, citrate, acetate, etc. in pH ranges of 2.5-3.5. However, the use of phosphate or other non-calcium chelating buffers allows the subsequent cHA column to be used repeatedly without exposure to chelating buffer. Product peak collection starts with the rise in UV absorbance and continues until the absorbance peak returns near to baseline. There are no critical peak cutting criteria. The intent is to collect the entire peak, while preventing unnecessary dilution of the product by collecting more eluate after UV absorbance returns close to baseline. For stability purposes, the recommended storage time for unadjusted MABSELECT™ eluate is up to 14 days at 2-10° C. Following elution, eluates are pooled, and immediately adjusted to pH 3.5. The MABSELECT™ column is stripped with a pH 1.5 hydrochloric acid solution between cycles, and is cleaned with 6 M guanidine in 50 mM sodium phosphate pH 7.0 at the end of the batch. The MABSELECT™ column is stored in 0.1M sodium acetate, 0.5 M sodium chloride containing 1% benzyl alcohol pH 5.2.

Multiple cycles may be necessary, depending on mass of product in the CUB.

MABSELECT™ chromatography removes a large portion of cell and media derived impurities and viral particles. In a novel approach, the salt wash is included to achieve additional DNA clearance. This affinity purification technique results ≧90% purity of the IgG with yields in excess of 90%.

### 2.2 pH Adjustment and Filtration

The pooled protein A eluate is adjusted to pH 2.5-4.5 with acid. Following a 15-60 minute hold for viral inactivation, eluate is adjusted to pH 6.0-8.5 with base. The pH adjusted eluate is then filtered to remove any precipitate, 0.2 um filtered.

The low pH treatment is intended to inactivate potential viral contaminants, especially retroviruses and other enveloped viruses. Filtration at higher pH prepares the solution for the next chromatography step and reduces DNA.

### 2.3 cHA Chromatography

Filtered eluate (from Step 2.2) is further purified by hydroxyapatite chromatography (using any hydroxyapatite resin such as BioRad CHT™ Type I resin or CHT™ Type II resin).

The column is prepared for chromatography by rinsing with 2-5 BV of pre-equilibration buffer consisting of 100 mM-1M phosphate, pH 6.0-8.5. (Alternative buffers to be considered for appropriate use throughout the process include but are not limited to Tris, acetate, MES, etc.) The column is then equilibrated with 2-10 BV of equilibration buffer consisting of 10-50 mM phosphate, 0-50 mM sodium chloride, pH 6.0-8.5. The cHA load sample is loaded onto the column. Upon completion of loading, the column is washed with 2-5 BV of Wash buffer consisting of 10-50 mM sodium phosphate, 0-50 mM sodium chloride, pH 6.5-8.0. Bound product is eluted with Elution Buffer consisting of 10-50 mM sodium phosphate, 50 mM-1.0 M sodium chloride, pH 6.5-8.5. The peak is collected when absorbance rises and continues until the peak returns to an absorbance corresponding to any pre-set criteria. Following eluate collection, the column is cleaned with 2-5 BV of 0.1-1N NaOH. Following cleaning the column is washed with 2-5 BV of 0.01-0.05N NaOH storage solution.

The cHA step simultaneously removes several impurities such as (but not limited to) non-IgG proteins, host cell proteins, DNA, protein A, protein A:IgG complexes, and viral particles.

### 2.4 Viral Filtration

The cHA Eluate (from Step 2.3) is filtered through a virus filter (of any type nominally quoted as capable of retaining particles equal to or greater then 20 nm in size including, but not limited to Pall DV20, Millipore VIRESOLVE® NFP, etc) virus removal filter according to manufacturers recommended procedure. Viral filtration will remove putative and/or actual viral contaminants.

### 2.5 Formulation by Ultrafiltration and Continuous Diafiltration

The filtrate (from Step 2.4) is diafiltered with 3-10 volumes of formulation buffer by tangential flow ultrafiltration (TFUF). The diafiltrate is then concentrated as necessary to a pre-determined concentration practical for human administration. After final concentration, the formulated bulk drug substance is 0.2 um filtered and stored at pre-determined temperatures.

### Example 3

### Includes Sequential Protein A to Anion Exchange to Ceramic Hydroxyapatite to Viral Filter and Final Formulation by Ultrafiltration/Diafiltration

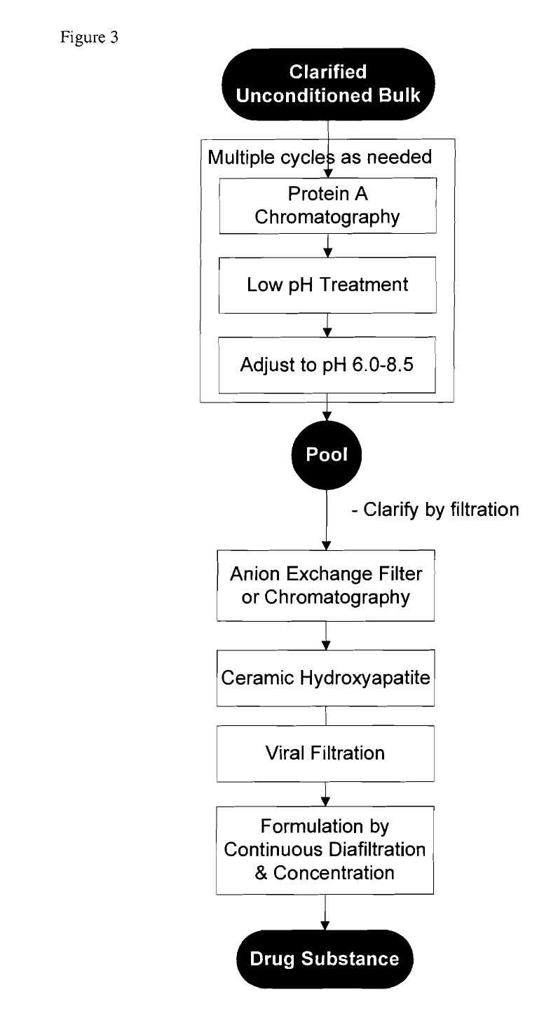

In another embodiment, the process involves monoclonal antibody purification by use of Protein A affinity, a low pH adjustment for viral inactivation, an additional preparative pH adjustment, anion exchange filtration (or chromatography), cHA chromatography, and final formulation by ultrafiltration/diafiltration. This process is depicted in FIG. 3.

An anion exchange filter (or column chromatography) is added as an example if additional clearance of DNA is need to give further assurance of product safety or quality.

Details of the individual process steps are described below. For each step, a brief description is given including the expected outcome. Important process parameters for each step are listed. All procedural details, buffer compositions, process set-points, column dimensions, etc. are included for illustrative purposes and should not be considered inclusive or restrictive in the operation of this art.

### 3.1 Affinity Chromatography

Monoclonal antibody (mAb, IgG) is captured from filtered cell culture media onto an affinity chromatography resin containing an immobilized recombinant Protein A ligand (including, but not limited to: Protein A SEPHAROSE™, recombinant Protein A SEPHAROSE™, MABSELECT™, MABSELECT SURE™, MABSELECT XTRA™, PROSEP® A, PROSEP® vA, PROSEP® rA, POROS® 50 A, AF-PROTEIN A TOYOPEARL® 650 M) which had previously been equilibrated with 10-50 mM phosphate, pH 6.0-8.5. Following a salt wash with 2-8 bed volumes (BV) of a 10-50 mM phosphate buffer containing 1-2M sodium chloride, in one embodiment 2M sodium chloride, pH 7.0 and a 3 BV wash with 20 mM sodium phosphate buffer pH 7.0, product is eluted with a pH shift using 10-50 mM sodium phosphate buffer, pH 2.1-3.5. Higher pH (e.g., up to pH 4.0) may be used if the yield is acceptable. One skilled in the art will recognize that buffer composition can include, but is not limited to phosphate, citrate, acetate, etc. in pH ranges of 2.5-3.5. However, the use of phosphate or other non-calcium chelating buffers allows the subsequent cHA column to be used repeatedly without exposure to chelating buffer. Product peak collection starts with the rise in UV absorbance and continues until the absorbance peak returns near to baseline. There are no critical peak cutting criteria. The intent is to collect the entire peak, while preventing unnecessary dilution of the product by collecting more eluate after UV absorbance returns close to baseline. For stability purposes, the recommended storage time for unadjusted MABSELECT™ eluate is up to 14 days at 2-10° C. Following elution, eluates are pooled, and immediately adjusted to pH 3.5. The MABSELECT™ column is stripped with a pH 1.5 hydrochloric acid solution between cycles, and is cleaned with 6 M guanidine in 50 mM sodium phosphate pH 7.0 at the end of the batch. The MABSELECT™ column is stored in 0.1M sodium acetate, 0.5 M sodium chloride containing 1% benzyl alcohol pH 5.2.

Multiple cycles may be necessary, depending on mass of product in the CUB.

MABSELECT™ chromatography removes a large portion of cell and media derived impurities and viral particles. In a novel approach, the salt wash is included to achieve additional DNA clearance. This affinity purification technique results ≧90% purity of the IgG with yields in excess of 90%.

### 3.2 pH Adjustment and Filtration

The pooled protein A eluate is adjusted to pH 2.5-4.5 with acid. Following a 15-60 minute hold for viral inactivation, eluate is adjusted to pH 6.0-8.5 with base. The pH adjusted eluate is then filtered to remove any precipitate, 0.2 um filtered.

The low pH treatment is intended to inactivate potential viral contaminants, especially retroviruses and other enveloped viruses. Filtration at higher pH prepares the solution for the next chromatography step and reduces DNA.

### 3.3 Anion Exchange Filtration or Chromatography

Filtered eluate (from Step 3.2) is further purified by filtration through an anion exchange filter (using any membrane with anion functionality including, but not limited to MUSTANG® Q or INTERCEPT™ Q) operated in a flow-through mode in which product is allowed to flow through the filter which had previously been equilibrated with buffer approximating that used for the elution from protein A column. In a minor variation, an anion exchange chromatography column (using any resin with a ligand of anion functionality including, but not limited to DEAE, Q-SEPHAROSE™, QXL, etc.) can be substituted for the filter and operated in a similar flow-through fashion. The anion exchange column is cleaned with 0.1-1 N NaOH and stored in 0.01-0.05N NaOH. The anion exchange step (filter or column version) further reduces the amount of DNA in the product.

### 3.4 cHA Chromatography

Filtered eluate (from Step 3.3) is further purified by hydroxyapatite chromatography (using any hydroxyapatite resin such as BioRad CHT™ Type I resin or CHT™ Type II resin).

The column is prepared for chromatography by rinsing with 2-5 BV of pre-equilibration buffer consisting of 100 mM-1M phosphate, pH 6.0-8.5. (Alternative buffers to be considered for appropriate use throughout the process include but are not limited to Tris, acetate, MES, etc.) The column is then equilibrated with 2-10 BV of equilibration buffer consisting of 10-50 mM phosphate, 0-50 mM sodium chloride, pH 6.0-8.5. The cHA load sample is loaded onto the column. Upon completion of loading, the column is washed with 2-5 BV of Wash buffer consisting of 10-50 mM phosphate, 0-50 mM sodium chloride, pH 6.5-8.0. Bound product is eluted with Elution Buffer consisting of 10-50 mM sodium phosphate, 50 mM-1.0 M sodium chloride, pH 6.5-8.5. The peak is collected when absorbance rises and continues until the peak returns to an absorbance corresponding to any pre-set criteria. Following eluate collection, the column is cleaned with 2-5 BV of 0.1-1N NaOH. Following cleaning the column is washed with 2-5 BV of 0.01-0.05N NaOH storage solution.

The cHA step simultaneously removes several impurities such as (but not limited to) non-IgG proteins, host cell proteins, DNA, protein A, protein A:IgG complexes, and viral particles.

### 3.5 Viral Filtration

The cHA Eluate (from Step 3.4) is filtered through a virus filter (of any type nominally quoted as capable of retaining particles equal to or greater then 20 nm in size including, but not limited to Pall DV20, Millipore VIRESOLVE® NFP, etc) virus removal filter according to manufacturers recommended procedure. Viral filtration will remove putative and/or actual viral contaminants.

### 3.6 Formulation by Ultrafiltration and Continuous Diafiltration

The filtrate (from Step 3.5) is diafiltered with 3-10 volumes of formulation buffer by tangential flow ultrafiltration (TFUF). The diafiltrate is then concentrated as necessary to a pre-determined concentration practical for human administration. After final concentration, the formulated bulk drug substance is 0.2 um filtered and stored at pre-determined temperatures.

### Example 4

### Includes sequential Protein A to Cation Exchange to MUSTANG® Q Filter to Ceramic Hydroxyapatite to Viral Filter and Final Formulation by Ultrafiltration/Diafiltration

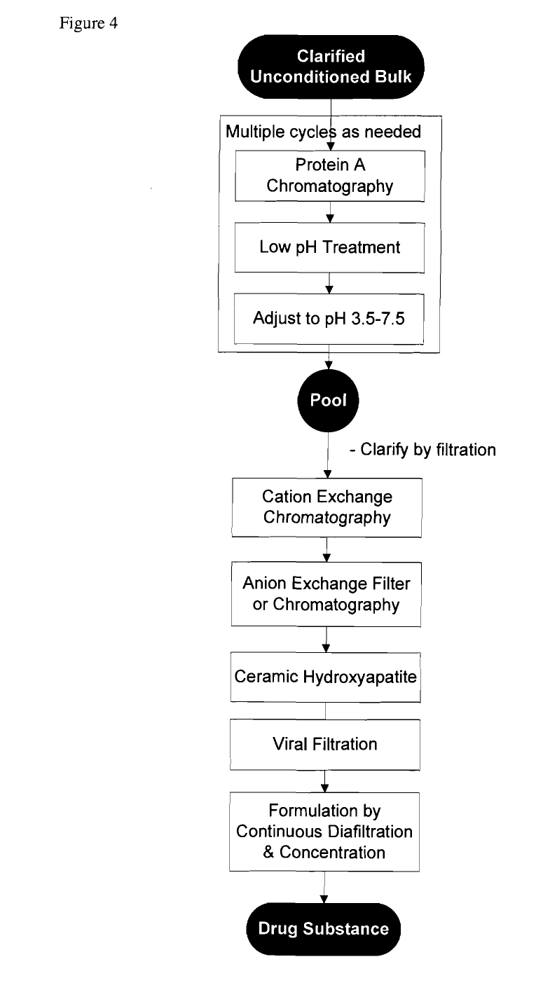

In another embodiment, the process involves monoclonal antibody purification by use of Protein A affinity, a low pH adjustment for viral inactivation, an additional preparative pH adjustment, cation exchange chromatography, anion exchange filtration (or chromatography), cHA chromatography, and final formulation by ultrafiltration/diafiltration. This process is depicted in FIG. 4.

The addition of an optional cation exchange step is added as an example if additional clearance of impurities is needed to give further assurance of product quality, purity, and/or safety.

Details of the individual process steps are described below. For each step, a brief description is given including an observed outcome. Representative process parameters for each step are listed. All procedural details, buffer compositions, process set-points, column dimensions, etc. are included for illustrative purposes and should not be considered inclusive or restrictive in the operation of this art.

### 4.1 Affinity Chromatography

Monoclonal antibody (mAb, IgG) is captured from filtered cell culture media onto an affinity chromatography resin containing an immobilized recombinant Protein A ligand (including, but not limited to: Protein A SEPHAROSE™, recombinant Protein A SEPHAROSE™, MABSELECT™, MABSELECT SURE™, MABSELECT XTRA™, PROSEP® A, PROSEP® vA, PROSEP® rA, POROS® 50 A, AF-PROTEIN A TOYOPEARL® 650 M) which had previously been equilibrated with 10-50 mM phosphate, pH 6.0-8.5. Following a salt wash with 2-8 bed volumes (BV) of a 10-50 mM phosphate buffer containing 1-2M, in one embodiment 2M sodium chloride pH 7.0 and a 3 BV wash with 20 mM sodium phosphate buffer pH 7.0, product is eluted with a pH shift using 10-50 mM citric acid buffer, or an integer within the range of 10-50 mM, in one embodiment 25 mM citric acid buffer, pH 2.5 to 4.5, in one embodiment pH 3.5. Product peak collection starts with the rise in UV absorbance and continues until the absorbance peak returns near to baseline. There are no critical peak cutting criteria. The intent is to collect the entire peak, while preventing unnecessary dilution of the product by collecting more eluate after UV absorbance returns close to baseline. For stability purposes, the recommended storage time for unadjusted MABSELECT™ eluate is up to 14 days at 2-10° C. Following elution, eluates are pooled, and immediately adjusted to pH 3.5. The MABSELECT™ column is stripped with a pH 1.5 hydrochloric acid solution between cycles, and is cleaned with 6 M guanidine in 50 mM sodium phosphate pH 7.0 at the end of the batch. The MABSELECT™ column is stored in 0.1M sodium acetate, 0.5 M sodium chloride containing 1% benzyl alcohol pH 5.2.

Multiple cycles may be necessary, depending on mass of product in the CUB.

MABSELECT™ chromatography removes a large portion of cell and media derived impurities and viral particles. In a novel approach, the salt wash (as described above with 1-2M sodium chloride pH 7.0 and 20 mM sodium phosphate buffer pH 7.0) is included to achieve additional DNA clearance. This affinity purification technique results in ≧90% purity of the IgG with yields in excess of 90%.

### 4.2 pH Adjustment and Filtration

The pooled protein A eluate is adjusted to pH 2.5-4.5 with acid. Following a 15-60 minute hold for viral inactivation, eluate is adjusted to pH 3.5-7.5 with base. The pH adjusted eluate is then 0.2 um filtered to remove any precipitate.

The low pH treatment is intended to inactivate potential viral contaminants, especially retroviruses and other enveloped viruses. Filtration at higher pH prepares the solution for the next chromatography step and reduces DNA.

### 4.3 Cation Exchange Chromatography

Filtered, pH-adjusted protein A eluate (from Step 4.2) is further purified by cation exchange chromatography (using a resin with a ligand of cation functionality including, but not limited to CM-SEPHAROSE™, SP-SEPHAROSE™, CM Hyper D, SPXL). The column is equilibrated with any buffer found to be compatible with product binding to cation exchange resin. One skilled in the art will recognize that buffer composition can include, but is not limited to phosphate, citrate, acetate, etc. in pH ranges of 2.5-7.0. However, the use of phosphate or other non-calcium chelating buffers allows for the subsequent cHA column to be used repeatedly without exposure to chelating buffer. After loading, the column is washed with 10-100 mM phosphate, pH 5.5-8.0. Product is eluted with 10-100 mM sodium phosphate, 10-200 mM sodium chloride, pH 5.5-8.0. The cation column is cleaned with 0.1-1 N NaOH and stored in 0.01-0.05N NaOH.

The cation exchange step removes additional protein and non-protein impurities including aggregate, viral particles, and DNA. This step also serves to buffer exchange the product into phosphate and avoids the use of citrate or other chelating buffers in cHA chromatography.

### 4.4 Anion Exchange Filtration or Chromatography

Filtered eluate (from Step 4.3) is further purified by filtration through an anion exchange filter (using any membrane with anion functionality including, but not limited to MUSTANG® Q or INTERCEPT™ Q) operated in a flow-through mode in which product is allowed to flow through the filter which had previously been equilibrated with buffer approximating that used for the elution from cation exchange column. In a minor variation, an anion exchange chromatography column (using any resin with a ligand of anion functionality including, but not limited to DEAE, Q-SEPHAROSE™, QXL, etc.) can be substituted for the filter and operated in a similar flow-through fashion. The anion exchange column is cleaned with 0.1-1 N NaOH and stored in 0.01-0.05N NaOH. The anion exchange step (filter or column version) further reduces the amount of DNA and viral particles in the product.

### 4.5 cHA Chromatography

Filtered eluate (from Step 4.4) is further purified by hydroxyapatite chromatography (using a hydroxyapatite resin including, but not limited to BioRad ceramic hydroxyapatite CHT™ Type I resin or CHT™ Type II resin).

The column is prepared for chromatography by rinsing with 2-5 BV of pre-equilibration buffer consisting of 100 mM-1M phosphate, pH 6.5-8.5. (Alternative buffers to be considered for appropriate use throughout the process include but are limited to Tris, acetate, MES, etc.) The column is then equilibrated with 2-10 BV of equilibration buffer consisting of 10-50 mM phosphate, 0-50 mM sodium chloride, pH 6.5-8.5. The cHA load sample is loaded onto the column. Upon completion of loading, the column is washed with 2-5 BV of Wash buffer consisting of 10-50 mM phosphate, 0-50 mM sodium chloride, pH 6.5-8.5. Bound product is eluted with Elution Buffer consisting of 10-50 mM sodium phosphate, 50 mM-1.0 M sodium chloride, pH 6.5-8.5. The peak is collected when absorbance rises and continues until the peak returns to an absorbance corresponding to any pre-set criteria. Following eluate collection, the column is cleaned with 2-5 BV of 0.1-1N NaOH. Following cleaning the column is washed with 2-5 BV of 0.01-0.05N NaOH storage solution.

The cHA step simultaneously removes several impurities such as (but not limited to) non-IgG proteins, host cell proteins, DNA, protein A, and protein A:IgG complexes.

### 4.6 Viral Filtration

The cHA eluate (from Step 4.5) is filtered through a virus filter (of any type nominally quoted as capable of retaining particles equal to or greater then 20 nm in size including, but not limited to Pall DV20, Millipore VIRESOLVE® NFP, etc) virus removal filter according to manufacturers recommended procedure. Viral filtration will remove putative and/or actual viral contaminants.

### 4.7 Formulation by Ultrafiltration and Continuous Diafiltration

The filtrate (from Step 4.6) is diafiltered with 3-10 volumes of formulation buffer by tangential flow ultrafiltration (TFUF). The diafiltrate is then concentrated as necessary to a pre-determined concentration suitable for human administration. After final concentration, the formulated bulk drug substance is 0.2 um filtered and stored at pre-determined temperatures.

### Example 5

### Purification of Monoclonal Antibody (an IgG₁) with Cation Exchange Included

In another embodiment, the process involves purification of an IgG₁ monoclonal antibody through protein A affinity, pH adjustment, cation exchange chromatography, and cHA chromatography with high yield and demonstrating significant removal of product aggregate, DNA, host cell protein and residual protein A.

### 5.1 MabSelect Affinity Chromatography

A monoclonal antibody (hereafter referred to as “IgG₁”) was captured from 0.3 um depth-filtered CUB onto a 0.5 cm×20 cm column of MABSELECT™ (GE Healthcare) affinity chromatography resin. The MABSELECT™ column was first equilibrated with 5 BV of 20 mM sodium phosphate, pH 7.0. The CUB was then loaded onto the column. Following a wash with 3 BV of a 20 mM phosphate buffer containing 2M sodium chloride pH 7.0, and a 3 BV wash with 20 mM sodium phosphate buffer pH 7.0, product was eluted with a pH shift using 25 mM citric acid buffer, pH 3.5. Product peak collection started with the rise in UV absorbance and continued until the absorbance peak returned near to baseline. Following elution, the eluate was immediately adjusted to pH 3.5. The MABSELECT™ column was stripped with a pH 1.5 hydrochloric acid solution and was cleaned with 6 M guanidine in 50 mM sodium phosphate pH 7.0. The MABSELECT™ column was stored at 2-8° C. in 0.1M sodium acetate, 0.5 M sodium chloride containing 1% benzyl alcohol pH 5.2.

### 5.2 pH 3.5 Adjustment and pH 5.0 Filtration

The pooled MABSELECT™ eluate (from step 5.1) was adjusted to pH 3.5 with 2.5 M HCl. Following a 30 minute hold (viral inactivation), eluate was adjusted to pH 5.0 with 3 M Tris base. The pH 5.0 eluate was depth filtered to remove any precipitate, 0.2 um filtered and held at 2-8 C until further processing. Storage temperature may also occur at room temperature.

### 5.3 SP-Sepharose Fast Flow Chromatography

Filtered MABSELECT™ eluate at pH 5.0 (from step 5.2) was further purified by cation exchange chromatography using SP-SEPHAROSE FF (GE Healthcare). A 0.5 cm×20 cm column of SP-SEPHAROSE Fast Flow (SPSFF) cation exchange chromatography resin was used. The SPSFF column was first equilibrated with 5 BV of 30 mM Sodium Citrate pH 5.0. The MABSELECT™ eluate was then loaded onto the column. Following loading and washing with 30 mM sodium phosphate, pH 7.0, product was eluted with 30 mM sodium phosphate, 120 mM sodium chloride, pH 7.0. Product peak collection started as UV absorbance increased and continued until peak absorbance returns to approximately 3% maximum peak height. SP-SEPHAROSE FF eluate was 0.2 um filtered. The SP-SEPHAROSE FF column was cleaned with 0.5 N NaOH and stored in 0.01 N NaOH.

### 5.4 cHA Chromatography

Filtered QSFF eluate (from step 5.3) was further purified by ceramic hydroxyapatite chromatography using cHA Type I resin (Bio-Rad). The column was prepared for chromatography by rinsing with ≧5 BV of 400 mM sodium phosphate, pH 6.8 pre-equilibration buffer. The column was then equilibrated with 5 BV of 10 mM sodium phosphate, 50 mM sodium chloride, pH 6.8 equilibration buffer. The cHA load sample was loaded onto the column. Upon completion of loading, the column was washed with 5 BV of 10 mM sodium phosphate, 50 mM sodium chloride, pH 6.8 wash buffer. Following loading and washing, product was eluted with 10 mM sodium phosphate, 1.0 M sodium chloride, pH 7.8. Product peak collection started as UV absorbance increased and continued until the peak returned to an absorbance corresponding to 3.0 mg/mL. The cHA eluate was 0.2 um filtered. Following eluate collection, the column was cleaned with 5 BV of 0.5 N NaOH. Following cleaning the column was washed with 3 BV of 0.01 N NaOH storage solution and the column stored at 18-25° C.

### 5.5 Analytical Testing

Samples taken throughout the purification described in 4.1-4.4 were tested for concentration by A280, % product aggregation, residual proteinA content, host cell protein, and host cell DNA. In addition, the procedure described above was repeated on new columns, with each load sample spiked with model virus, and tested for clearance through each step in accordance with ICH guidelines. The results of this testing is compiled in Table 2.

TABLE 2
Example 4 Process Performance

| | Yield | Aggregate | Protein A | HCP | DNA |
|---|---|---|---|---|---|
| Step | (%) | (%) | (ng/mg) | (ppm) | (pg/mg) |
| MabSelect Eluate | 92 | 1.1 | 3.5 | 8.9 | 420 |
| pH 3.5 hold and pH | 91 | 1.2 | 2.0 | 3.3 | 4.0 |
| 5.0 Filtration | | | | | |
| SPSFF Eluate | 100 | 1.3 | <1.4 | <0.6 | 0.9 |
| cHA Eluate | 83 | <0.1 | <0.2 | <0.4 | <0.08 |

### Example 6

### Purification of Monoclonal Antibody (an IgG₁) with Cation and Anion Exchange Included

In another embodiment, the process involves purification of an IgG₁ monoclonal antibody through protein A affinity, pH adjustment, cation exchange chromatography, anion exchange chromatography, and cHA chromatography with high yield and demonstrating significant removal of product host cell protein and residual protein A.

### 6.1 MabSelect Affinity Chromatography

A monoclonal antibody (hereafter referred to as “IgG₁”) was captured from 0.3 um depth-filtered CUB onto a 30.0 cm×20 cm column of MABSELECT™ (GE Healthcare) affinity chromatography resin. The MABSELECT™ column was first equilibrated with 5 BV of 20 mM sodium phosphate, pH 7.0. The CUB was then loaded onto the column. Following a wash with 3 BV of a 20 mM phosphate buffer containing 2M sodium chloride pH 7.0, and a 3 BV wash with 20 mM sodium phosphate buffer pH 7.0, product was eluted with a pH shift using 25 mM citric acid buffer, pH 3.5. Product peak collection started with the rise in UV absorbance and continued until the absorbance peak returned near to baseline. Following elution, the eluate was immediately adjusted to pH 3.5. The MABSELECT™ column was stripped with a pH 1.5 hydrochloric acid solution and was cleaned with 6 M guanidine in 50 mM sodium phosphate pH 7.0. The MABSELECT™ column was stored at 2-8° C. in 0.1M sodium acetate, 0.5 M sodium chloride containing 1% benzyl alcohol pH 5.2.

### 6.2 pH 3.5 Adjustment and pH 5.0 Filtration

The pooled MABSELECT™ eluate (from step 6.1) was adjusted to pH 3.5 with 2.5 M HCl. Following a 30 minute hold (viral inactivation), eluate was adjusted to pH 5.0 with 3 M Tris base. The pH 5.0 eluate was depth filtered to remove any precipitate, 0.2 um filtered and held at 2-8 C until further processing. Storage may also occur at room temperature.

### 6.3 SP-Sepharose Fast Flow Chromatography

A sample of Filtered MABSELECT™ eluate at pH 5.0 (from step 6.2) was further purified by cation exchange chromatography using SP-SEPHAROSE FF (GE Healthcare). A 4.4 cm×20 cm column of SP-SEPHAROSE Fast Flow (SPSFF) cation exchange chromatography resin was used. The SPSFF column was first equilibrated with 5 BV of 30 mM Sodium Citrate pH 5.0. The MABSELECT™ eluate was then loaded onto the column. Following loading and washing with 30 mM sodium phosphate, pH 7.0, product was eluted with 30 mM sodium phosphate, 120 mM sodium chloride, pH 7.0. Product peak collection started as UV absorbance increased and continued until peak absorbance returns to approximately 3% maximum peak height. SP-SEPHAROSE FF eluate was 0.2 um filtered. The SP-SEPHAROSE FF column was cleaned with 0.5 N NaOH and stored in 0.01 N NaOH.

### 6.4 Q-Sepharose Fast Flow Chromatography

Filtered SP-SEPHAROSE FF eluate (from step 6.3) was further purified by chromatography on 4.4×10 cm Q-SEPHAROSE Fast Flow (QSFF). The chromatography was operated in a flow-through mode in which SPSFF eluate was allowed to flow through the QSFF column. The column was first prepared by washing with 3 CV of water, and a salt wash consisting of 3 CV of 40 mM Tris, 2M NaCl, pH 8.0. The SPSFF eluate was then loaded onto the column and product peak collection started as UV absorbance increased and continued until peak absorbance returned to approximately 3% maximum peak height. QSFF eluate was 0.2 um filtered. The QSFF column was cleaned with 0.5 M NaOH and stored in 0.01 M NaOH.

### 6.5 cHA Chromatography

Filtered QSFF eluate (from step 6.4) was further purified by ceramic hydroxyapatite chromatography using cHA Type I resin (Bio-Rad). The 0.5 cm×20 cm column was prepared for chromatography by rinsing with ≧5 BV of 400 mM sodium phosphate, pH 6.8 pre-equilibration buffer. The column was then equilibrated with 5 BV of 30 mM sodium phosphate, pH 7.0 equilibration buffer. The cHA load sample was loaded onto the column. Upon completion of loading, the column was washed with 5 BV of 30 mM sodium phosphate, pH 7.0 wash buffer. Following loading and washing, product was eluted with 30 mM sodium phosphate, 500 mM sodium chloride, pH 7.5. Product peak collection started as UV absorbance increased and continued until the peak returned to approximately 10% maximum peak height. The cHA eluate was 0.2 um filtered. Following eluate collection, the column was cleaned with 5 BV of 0.5 N NaOH. Following cleaning the column was washed with 3 BV of 0.01 N NaOH storage solution and the column stored at 18-25° C.

### 6.6 Analytical Testing

Samples taken throughout the purification described in 6.1-6.5 were tested for concentration by A280. Samples of the cHA load and eluate were tested for residual protein A content, and host cell protein. The results of this testing is compiled in Table 3

TABLE 3
Example 5 Process Performance

| | Yield | Protein A | HCP |
|---|---|---|---|
| Step | (%) | (ng/mg) | (ppm) |
| MABSELECT ™ | 100 | NM* | NM |
| Eluate | | | |
| pH 3.5 hold and pH | 73 | NM | NM |
| 5.0 Filtration | | | |
| SPSFF Eluate | 97 | NM | NM |
| QSFF Eluate | 103 | 0.5 | ≧11.3 |
| cHA Eluate | 91 | <0.03 | 0.5 |

*NM = Not Measured

### Example 7

### Purification of Monoclonal Antibody (an IgG₁) Showing Viral Clearance

In another embodiment, the process involves purification of IgG₁ through protein A affinity, pH adjustment, cation exchange chromatography, anion exchange chromatography, cHA chromatography, and DV20 filtration, while demonstrating significant removal of 2 model viruses, xenotropic Murine Leukemia Virus (xMuLV) and Porcine Parvovirus (PPV).

### 7.1 MabSelect Affinity Chromatography

A monoclonal antibody (hereafter referred to as “IgG₁”) was captured from 0.3 um depth-filtered virus-spiked CUB onto a 0.5 cm×20 cm column of MABSELECT™ (GE Healthcare) affinity chromatography resin. The MABSELECT™ column was first equilibrated with 5 BV of 20 mM sodium phosphate, pH 7.0. The CUB was then loaded onto the column. Following a wash with 3 BV of a 20 mM phosphate buffer containing 2M sodium chloride pH 7.0, and a 3 BV wash with 20 mM sodium phosphate buffer pH 7.0, product was eluted with a pH shift using 25 mM citric acid buffer, pH 3.5. Product peak collection started with the rise in UV absorbance and continued until the absorbance peak returned near to baseline. Following elution, the eluate was immediately adjusted to pH 3.5. The MABSELECT™ column was stripped with a pH 1.5 hydrochloric acid solution and was cleaned with 6 M guanidine in 50 mM sodium phosphate pH 7.0. The MABSELECT™ column was stored at 2-8° C. in 0.1M sodium acetate, 0.5 M sodium chloride containing 1% benzyl alcohol pH 5.2.

### 7.2 pH 3.5 Adjustment and pH 5.0 Filtration

Virus-spiked MABSELECT™ eluate was adjusted to pH 3.5 with 2.5 M HCl. Following a 30 minute hold (viral inactivation), eluate was adjusted to pH 5.0 with 3 M Tris base. The pH 5.0 eluate was depth filtered to remove any precipitate, 0.2 um filtered and held at 2-8 C until further processing.

### 7.3 SP-Sepharose Fast Flow Chromatography

Virus-spiked Filtered MABSELECT™ eluate at pH 5.0 was purified by cation exchange chromatography using SP-SEPHAROSE FF (GE Healthcare). A 0.5 cm×20 cm column of SP-SEPHAROSE Fast Flow (SPSFF) cation exchange chromatography resin was used. The SPSFF column was first equilibrated with 5 BV of 30 mM Sodium Citrate pH 5.0. The MABSELECT™ eluate was then loaded onto the column. Following loading and washing with 30 mM sodium phosphate, pH 7.0, product was eluted with 30 mM sodium phosphate, 120 mM sodium chloride, pH 7.0. Product peak collection started as UV absorbance increased and continued until peak absorbance returns to approximately 3% maximum peak height. SPSFF eluate was 0.2 um filtered. The SP-SEPHAROSE FF column was cleaned with 0.5 N NaOH and stored in 0.01 N NaOH.

### 7.4 Q-Sepharose Fast Flow Chromatography

Virus-spiked Filtered SP-SEPHAROSE FF eluate was purified by chromatography on 0.5×10 cm Q-SEPHAROSE Fast Flow (QSFF). The chromatography was operated in a flow-through mode in which SPSFF eluate was allowed to flow through the QSFF column. The column was first prepared by washing with 3 CV of water, and a salt wash consisting of 3 CV of 40 mM Tris, 2M NaCl, pH 8.0. The SPSFF eluate was then loaded onto the column and product peak collection started as UV absorbance increased and continued until peak absorbance returned to approximately 3% maximum peak height. QSFF eluate was 0.2 um filtered. The QSFF column was cleaned with 0.5 M NaOH and stored in 0.01 M NaOH.

### 7.5 cHA Chromatography

Virus-spiked filtered QSFF eluate was purified by ceramic hydroxyapatite chromatography using cHA Type I resin (Bio-Rad). The 0.5 cm×20 cm column was prepared for chromatography by rinsing with ≧5 BV of 400 mM sodium phosphate, pH 6.8 pre-equilibration buffer. The column was then equilibrated with 5 BV of 10 mM sodium phosphate, 50 mM sodium chloride, pH 6.8 equilibration buffer. The cHA load sample was loaded onto the column. Upon completion of loading, the column was washed with 5 BV of 10 mM sodium phosphate, 50 mM sodium chloride, pH 6.8 wash buffer. Following loading and washing, product was eluted with 10 mM sodium phosphate, 1.0 M sodium chloride, pH 7.8. Product peak collection started as UV absorbance increased and continued until the peak returned to an absorbance corresponding to 3.0 mg/mL. The cHA eluate was 0.2 um filtered. Following eluate collection, the column was cleaned with 5 BV of 0.5 N NaOH. Following cleaning the column was washed with 3 BV of 0.01 N NaOH storage solution and the column stored at 18-25° C.

### 7.6 Viral Filtration (DV20)

Virus-spiked cHA Eluate was filtered through a Pall DV20 virus removal filter according to manufacturers recommended procedure. DV20 filtrate was 0.2 um filtered and held at 2-8° C.

DV20 filtration will remove putative and/or actual viral contaminants. Size exclusion removal of viruses as small as 20 nm is claimed by the manufacturer.

### 7.7 Analytical Testing

Samples taken throughout the purification were tested for clearance through each step in accordance with ICH guidelines. The log reduction values (LRV) resulting from this testing are compiled in Table 3.

TABLE 3
Process Performance

| Step | MuLV LRV | PPV LRV |
|---|---|---|
| MabSelect Eluate | >6.41 | 3.29 |
| pH 3.5 hold and pH | >6.97 | NM |
| 5.0 Filtration | | |
| SPSFF Eluate | 1172.70 | 1.59 |
| QSFF Eluate | >5.06 | 2.60 |
| cHA Eluate | >4.62 | 2.65 |
| DV20 | >5.55 | 2.83 |

All documents cited herein and patent applications to which priority is claimed are incorporated by reference herein in their entirety. This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

What is claimed is:

1. A method of purifying antibody comprising:
a) contacting an antibody-containing solution to ceramic hydroxyapatite chromatography media, wherein the antibody is adsorbed by the ceramic hydroxyapatite chromatography media; and
b) selectively eluting the antibody by isocratic elution that simultaneously removes at least one impurity.

2. The method according to claim 1, wherein the said at least one impurities are selected from the group of: host cell proteins, host cell nucleic acids, retroviral particles, adventitious viruses, impurities introduced during production, impurities introduced during purification, and aggregated forms of the antibody.

3. The method according to claim 1, wherein the antibody is selected from the group of: a monoclonal antibody, a fragment of monoclonal antibody, a polyclonal antibody, and a fragment of polyclonal antibody.

4. A method of purifying a monoclonal antibody in an aqueous solution comprising the steps of:
a) contacting the monoclonal antibody in an aqueous solution to an affinity chromatography resin, wherein the monoclonal antibody is adsorbed by the affinity chromatography resin;
b) eluting the monoclonal antibody from the affinity chromatography resin;
c) inactivating viral contaminants by adjusting the monoclonal antibody eluate from step b) to pH 2.5 to 4.5;
d) adjusting the monoclonal antibody eluate from step c) to pH 6.0 to 8.5;
e) filtering the monoclonal antibody eluate from step d) through a 0.2 um filter;
f) filtering the monoclonal antibody filtrate from step e) through an anion exchange filter or anion exchange chromatography medium;
g) applying the monoclonal antibody filtrate from step f) to ceramic hydroxyapatite, wherein the monoclonal antibody is adsorbed by the ceramic hydroxyapatite; and
h) eluting the monoclonal antibody with an isocratic elution buffer.

5. The method of claim 4 further comprising the steps of:
i) filtering the monoclonal antibody eluate from step h) through a virus filter; and
j) formulating the monoclonal antibody from step i) by ultrafiltration and continuous diafiltration.

6. A method of purifying a monoclonal antibody or fragment thereof in an aqueous solution comprising the steps of:
a) contacting the monoclonal antibody or fragment thereof in an aqueous solution to an affinity chromatography resin containing an immobilized recombinant Protein A ligand, wherein the monoclonal antibody or fragment thereof is adsorbed by the affinity chromatography resin;
b) washing the monoclonal antibody or fragment thereof from step a) with a wash solution at about pH 7, which does not elute the monoclonal antibody or fragment thereof;
c) eluting the monoclonal antibody or fragment thereof from step b) with an elution buffer at pH 2.1 to 4.0;
d) inactivating viral contaminants by adjusting the monoclonal antibody or fragment thereof eluate from step c) to pH 2.5 to 4.5 with acid;
e) adjusting the monoclonal antibody or fragment thereof eluate from step d) to pH 6.0 to 8.5 with base;
f) filtering the monoclonal antibody or fragment thereof eluate from step e) through a 0.2 um filter;
g) binding the monoclonal antibody or fragment thereof from step f) to ceramic hydroxyapatite;
h) washing the bound monoclonal antibody or fragment thereof from step g) with a wash solution at pH 6.5 to 8.0, which does not elute the monoclonal antibody or fragment thereof;
i) eluting the monoclonal antibody or fragment thereof from step h) with an isocratic elution buffer at pH 6.5 to 8.0, 10 to 50 mM sodium phosphate and 50 mM to 1.0 M sodium chloride;
j) filtering the monoclonal antibody or fragment thereof eluate from step i) through a virus filter; and
k) formulating the monoclonal antibody or fragment thereof from step j) by ultrafiltration and continuous diafiltration.

7. A method of purifying a monoclonal antibody or fragment thereof in an aqueous solution comprising the steps of:
a) contacting the monoclonal antibody or fragment thereof in an aqueous solution to an affinity chromatography resin containing an immobilized recombinant Protein A ligand, wherein the monoclonal antibody or fragment thereof is adsorbed by the affinity chromatography resin;
b) washing the monoclonal antibody or fragment thereof from step a) with a wash solution at about pH 7, which does not elute the monoclonal antibody or fragment thereof;
c) eluting the monoclonal antibody or fragment thereof from step b) with an elution buffer at pH 2.1 to 4.0;
d) inactivating viral contaminants by adjusting the monoclonal antibody or fragment thereof eluate from step c) to pH 2.5 to 4.5 with acid;
e) adjusting the monoclonal antibody or fragment thereof eluate from step d) to pH 6.0 to 8.5 with base;
f) filtering the monoclonal antibody or fragment thereof eluate from step e) through a 0.2 um filter;
g) filtering the monoclonal antibody or fragment thereof eluate from step f) through an anion exchange filter or anion exchange chromatography medium;
h) binding the monoclonal antibody or fragment thereof from step g) to ceramic hydroxyapatite;
i) washing the bound monoclonal antibody or fragment thereof from step h) with a wash solution at pH 6.5 to 8.0, which does not elute the monoclonal antibody or fragment thereof;
j) eluting the monoclonal antibody or fragment thereof from step i) with an isocratic elution buffer at pH 6.5 to 8.0, 10 to 50 mM sodium phosphate and 50 mM to 1.0 M sodium chloride;
k) filtering the monoclonal antibody or fragment thereof eluate from step j) through a virus filter; and
l) formulating the monoclonal antibody or fragment thereof from step k) by ultrafiltration and continuous diafiltration.

8. A method of purifying a monoclonal antibody or fragment thereof in an aqueous solution comprising the steps of:
a) contacting the monoclonal antibody or fragment thereof in an aqueous solution to an affinity chromatography resin containing an immobilized recombinant Protein A ligand, wherein the monoclonal antibody is adsorbed by the affinity chromatography resin;
b) washing the monoclonal antibody or fragment thereof from step a) with a wash solution at about pH 7, which does not elute the monoclonal antibody or fragment thereof;
c) eluting the monoclonal antibody or fragment thereof from step b) with about 25 mM citric acid elution buffer at about pH 3.5;
d) inactivating viral contaminants by adjusting the monoclonal antibody or fragment thereof eluate from step c) to pH 2.5 to 4.5 with acid;
e) adjusting the monoclonal antibody or fragment thereof eluate from step d) to pH 3.5 to 7.5 with base;
f) filtering the monoclonal antibody or fragment thereof eluate from step e) through a 0.2 um filter;
g) binding the monoclonal antibody or fragment thereof eluate from step f) to a cation exchange chromatography medium;
h) washing the bound monoclonal antibody or fragment thereof from step g) with a wash solution at pH 5.5 to 8.0, which does not elute the monoclonal antibody or fragment thereof;
i) eluting the monoclonal antibody or fragment thereof from step h) with an elution buffer at pH 5.5 to 8.0, consisting of 10 to 100 mM sodium phosphate and 10 mM to 200 mM sodium chloride;
j) filtering the monoclonal antibody or fragment thereof eluate from step i) through an anion exchange filter or anion exchange chromatography medium;
k) binding the monoclonal antibody or fragment thereof from step j) to ceramic hydroxyapatite;
l) washing the bound monoclonal antibody or fragment thereof from step k) with a wash solution at pH 6.5 to 8.0, which does not elute the monoclonal antibody or fragment thereof;
m) eluting the monoclonal antibody or fragment thereof from step 1) with an isocratic elution buffer at pH 6.5 to 8.0, 10 to 50 mM sodium phosphate and 50 mM to 1.0 M sodium chloride;
n) filtering the monoclonal antibody or fragment thereof eluate from step m) through a virus filter; and
o) formulating the monoclonal antibody or fragment thereof from step n) by ultrafiltration and continuous diafiltration.
